(19) United States
(12) Patent Application Publication
(10) Pub. No.: US 2022/0084689 A1
(43) Pub. Date: Mar. 17, 2022

(54) SYSTEM AND METHOD FOR IDENTIFICATION OF AN ADEQUATE HEALTHCARE AGREEMENT ACCORDING TO A GIVEN MEDICAL CONDITION

(71) Applicant: MEDIWHO LTD, Haifa (IL)

(72) Inventor: MICHAEL KOPELL, KIRIAT ATA (IL)

(21) Appl. No.: 17/533,231

(22) Filed: Nov. 23, 2021

Related U.S. Application Data
(63) Continuation of application No. PCT/IL2020/050349, filed on Mar. 24, 2020.
(60) Provisional application No. 62/854,327, filed on May 30, 2019.

(51) Int. Cl. G16H 70/60

(52) U.S. Cl. CPC G16H 70/60

(57) ABSTRACT

A method operable on a computerized system for matching privilege and optional treatment to medical conditions according to healthcare agreements, the method including multiple steps to match the privileges and the optional treatment to a person, including a step of generating a digital output including the medical condition of the person, and the course of actions to which the person is entitled, according to an identified relevant sections in the healthcare agreement portfolio, where the healthcare agreement portfolio is associated with the person's name, and a step of receiving a medical condition of a person represented as a record of data, where the record of data includes the medical condition thereof, and where the medical condition states of one or more aspects of the person's health.

### FIELD OF THE INVENTION

The present invention relates to a method and system for associating privilege according to a person's healthcare agreements and healthcare insurance, in particular to identify the coverage and the optional covered procedures & treatments to which the person is entitled.

### BACKGROUND OF THE INVENTION

People can have variety of healthcare insurances and healthcare agreements which detail the person's rights in various circumstances. The healthcare insurances and healthcare agreements are complex and common users cannot extract their rights from standard healthcare insurances and healthcare agreements they have. In many cases, people have multiple insurance agreements, for example from multiple companies or one insurance agreement via an employment agreement and another insurance agreement which is bought by the user. Multiple insurance agreements may result in a situation in which the person pays twice for the same insurance coverage, and wastes his/her money. In addition, many persons who sign the insurance agreements do not fully understand their medical coverage, as there are thousands of medical situations that may be covered by the insurance agreements. Understanding the person's medical coverage requires much time and energy, in reviewing lengthy insurance agreements and understanding the legal and medical terms which are not clear to the common person.

Thus, the process of identifying the healthcare coverage a person has requires reviewing multiple documents and agreements. In multiple cases, there is no one place functioning as a focal point for all documents and agreements. Furthermore, even a person could consolidate all the agreements, the documents the signed documents and the memorandums the person has received, identify the coverage the medical treatments, the cost and the required operation, involves with knowledge in multiple areas. Such areas can be law, insurance, and the like. In some cases, the coverage and the optional medical treatments to which the person is entitled is associated with a specific health condition.

### SUMMARY OF THE INVENTION

The present disclosure discloses a method operable on a computerized system for matching privilege and optional treatment to medical conditions according to healthcare agreements. Such a method comprises multiple steps to match the privileges and the optional treatment to a person. The method disclosed herein can also comprise a step of generating a digital output comprising the medical condition of the person, and the course of actions to which the person is entitled, according to an identified relevant sections in the healthcare agreement portfolio, wherein the healthcare agreement portfolio is associated with the person's name. Thus, such a method can comprise a step of receiving a medical condition of a person represented as a record of data, wherein the record of data comprises the medical condition thereof, and wherein the medical condition states of one or more aspects of the person's health.

The method disclosed herein can also comprise a step of categorizing the record of data representing the medical condition, wherein the categorizing process categorizes terms, and/or phrases, and/or word which appear in the provided record of data representing the medical condition. The categorization process can be followed by a step of identifying a healthcare agreement portfolio associated with the person associated with the provided record of data representing the medical condition. In such cases, wherein the categorization process is completed, the computerized system can also be configured to identify the relevant sections in the healthcare agreement portfolio which are related to the categories identified in the record of data representing the medical condition.

The method disclosed herein can also comprise a step of identifying the course of actions to which the person is entitled, according to the identified relevant sections in the healthcare agreement portfolio which are related to the categories identified in the record of data representing the medical condition thereof. In case the course of actions which the person is entitled are identified, the computerized system can generate a digital output comprising the person's name, the categories associated to the medical condition of the person, and the course of actions to which the person is entitled, according to the identified relevant sections in the healthcare agreement portfolio.

In some cases, computerized system can also be configured to associate the covered cases, corresponding with the identified healthcare agreement portfolio. In such cases, the covered cases are sets of at least one course of action. In some cases, the computerized system can also perform a classifying process to identify classes in the record of data representing the medical condition to classes. In such cases, the classes can be identified sets of terms having some property or attribute in common and differentiated from others by kind, type, or quality.

The subject matter also discloses receiving personal information from a user of the computerized system and suggesting personal medical insurance coverage to the specific person. The personal medical insurance coverage is calculated according to the personal and medical information inputted by the user into the computerized system. Such personal information may be the person's age, gender, place of residence, salary and the like the medical information may include smoking habits, nutrition habits, prior surgeons the user had, prior diseases and/or medical conditions the user suffered from, medical drugs consumed regularly by the user and the like. Then, the computerized system compares the person's medical and personal information to a benchmark which represents the specific person's medical and personal information. Such benchmark may comprise multiple medical insurance policies and/or coverages of other persons having similar characteristics to the specific person, for example having a similarity of more than 80 percent to the medical information inputted by the user. Then, the computerized system reviews the specific person's medical insurance coverage and compares it to the benchmark. In case the benchmark has more coverage, the system may suggest the specific person to but the difference between the person's current coverage and the benchmark coverage. In case the user has more coverage than the benchmark coverage, the system may suggest the person to replace and/or correct his/her medical insurance agreement. In some cases, the specific person may select a level of benchmark, for example basic, normal and high, which indicate a level of medical coverage. For example, a high benchmark will have more coverage for a specific age and gender than the standard benchmark.

The computerized system may comprise a module in which the users input financial information, such as invoices and/or receipts that indicate payment for medical services, such as appointments, medical diagnosis, medical devices, medical drugs, surgeries and the like. The users may input additional information, such as the date in which the payment was made. The system will then review the users' medical insurance coverage and issue a request for reimbursement for the relevant entity that is entitled to reimburse the user, such as the insurance company.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows a logical structure manifesting the reciprocal relations between healthcare agreement portfolios and optional courses of actions corresponding thereof, according to exemplary embodiments of the present invention;

FIG. 2 discloses a computerized system for associating course of actions to medical conditions based on personal healthcare agreement portfolio, according to exemplary embodiments of the present invention;

FIG. 3 shows a method configured to receive medical condition representation comprising a person name and a medical condition thereof and generating an output comprising the optional courses of actions and the covered cases of the healthcare agreement portfolio, according to exemplary embodiment of the present invention;

FIG. 4 shows a registration process of a person, to the computerized system, according to exemplary embodiments of the present invention, and;

FIG. 5 shows a process of identifying covered cases in a healthcare agreement portfolio, according to exemplary embodiments of the present invention.

### DETAILED DESCRIPTION OF THE INVENTION

According to possible embodiments of the system and method disclosed herein, a medical condition associated with a person can be linked to at least one course of actions. In some cases, the at least one course of actions is predefined in the system, for the purposes of addressing the medical condition associated with the person. In some embodiments of the disclosed system and method, the at least one course of actions is corresponded with agreements or agreements' clauses of healthcare agreement portfolio. Such a healthcare agreement portfolio can be associated with the person associated with said medical condition.

The term medical condition utilized herein depicts a state of one or more aspects of the health of a person. In some cases, the medical condition, also known as a medical state of the person can denote diseases, lesions, disorders, mental illnesses, illness, injury and/or nonpathological condition that normally receives medical treatment, such as pregnancy or childbirth. In some cases, the medical condition can denote a clinical status or any health-related status which requires a medical treatment. In some cases, the medical condition can be represented as a record of data, denoted herein as a medical condition representation, can comprise indicators, numeric values, textual descriptions, images, or any other evidence-based data indicating a medical condition of a person. In some cases, the medical condition representation can comprise a written document denoting the medical condition, or one aspect of the medical condition of a person. In some other cases, the medical condition representation can comprise a photographic evidence denoting the medical condition, or one aspect of the medical condition of a person. In some embodiments disclosed herein, the medical condition representation can comprise an auditory representation embodied as a computer-readable fashion.

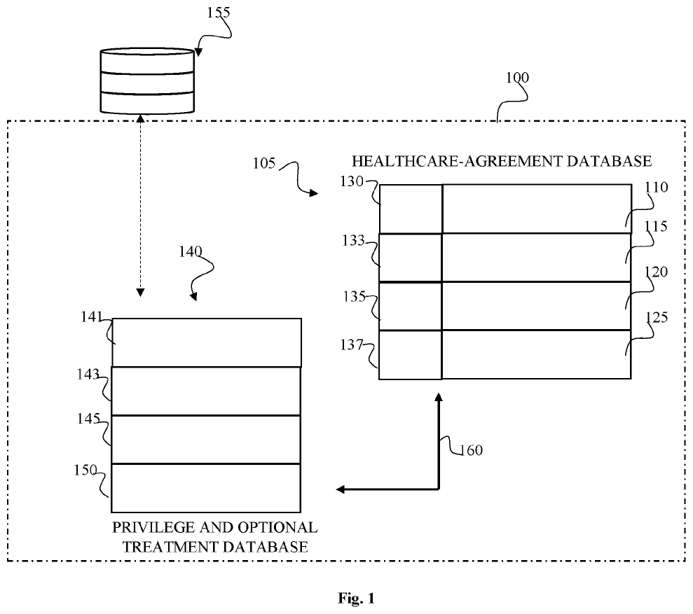

FIG. 1 shows a logical structure manifesting the reciprocal relations between healthcare agreement portfolios and optional courses of actions corresponding thereof, according to exemplary embodiments of the present invention. FIG. 1 shows a medical related information module 100 embodied as a logical structure which can comprise a healthcare-agreement database 105 designed to store healthcare agreement portfolio, associated with a person. The healthcare-agreement database 105 comprises agreement records such as agreement records 110, 115, 120, and 125. In some cases, agreement records 110, 115, 120, and 125 can be associated with one person. The agreement records 110, 115, 120, and 125 can comprise documented healthcare agreements, especially healthcare agreements which cover the whole or a part of events causing medical expenses.

In some cases the healthcare agreements held by the agreement records 110, 115, 120, and 125 can be such as, healthcare insurances, health agreements set out according to a National Health Insurance Law, health agreements set out according to a national social health insurance system, health agreements set out according to any healthcare system a person may have, health agreements set out according to any health service or universal healthcare system, social security act, applicable laws and the like. In some cases, the healthcare agreements held by the agreement records 110, 115, 120, and 125 can be such as healthcare agreements set out according to national programs such as Patient Protection and Affordable Care Act, or healthcare agreements financed by employers.

In some cases, a person may have more than one healthcare agreement held and stored by the healthcare-agreement database 105. For example, a person may have a private healthcare insurance signed with an insurance company, a healthcare agreement set out according to any universal healthcare system, and some and some entitlements for healthcare treatments provided by the employer of that person. In such cases, the healthcare-agreement database 105 can be configured to group the healthcare agreements associated with that person to a healthcare agreement portfolio. For example, the healthcare agreements of a person can be held and/or stored by the agreement records 110, 115 and grouped under one healthcare agreement portfolio associated with said person. Thus, the healthcare agreement portfolio can be a group of healthcare agreements associated with a person, wherein the healthcare agreement portfolio can be processed and utilized a whole. For example, the healthcare-agreement database 105 can be configured to define a healthcare agreement portfolio comprising more than one healthcare agreement associated with one person. Thus, a person can be associated with at least one healthcare agreement portfolio. In some cases, a person can be associated with more than one healthcare agreement portfolio, wherein the associated portfolios have diverse types. For example, a healthcare agreement portfolio can be a family healthcare agreement portfolio, wherein the family members have family level aggregated rights. The family healthcare agreement portfolio may also comprise elderly parents supported by the family. In some cases, the person associated with the healthcare agreement portfolios can be provided with choice to define a specific family member, or a group of family members as a specific healthcare agreement portfolio. In some embodiments of the healthcare-agreement database 105, the healthcare-agreement database 105 can comprise category records such as category records 130, 133, 135, and 137. Category records 130, 133, 135, and 137 can comprise metadata tags assigned to specific part in the agreement records. The metadata tags can comprise keywords or terms assigned to a piece of information within the healthcare agreement stored in the healthcare-agreement database 105. In some cases, the category records 130, 133, 135, and 137 can be associated with specific clause in a specific healthcare agreement. For example, the category record 130 can be associated with the healthcare agreement in agreement records 110. In such an exemplary case, the category record 130 can comprise metadata associated with specific information within the healthcare agreement in agreement records 110. In some other cases, alternative metadata system may be utilized to identify specific terms and conditions, within the healthcare-agreement database 105.

The medical related information module 100 can also comprise a course of action database 140 designed and configured to store records 141, 143, 145, 150 of courses of actions corresponding with the healthcare agreement portfolio stored in the healthcare-agreement database 105. In some cases, such courses of actions stored in the course of action database 140 can be a digital link or a documented reference to healthcare institutes providing healthcare services. Such healthcare institutes can be a clinic, a hospital, an emergency room, a health maintenance organization, and the like. In some cases, such course of actions can also be a list of references of healthcare institutes providing similar services. In such cases, the medical related information module 100 may be configured to list similar services and associate them with a healthcare agreement portfolio of a person.

In some embodiments of the disclosed medical related information module 100, the course of action database 140 may associates data related to rules and regulations. For example, a person associated with a healthcare agreement portfolio can be entitled to receive a specific treatment and/or a specific medicine, according to the coverages of one or more healthcare agreements in the healthcare agreement portfolio. In such exemplary case, the course of action database 140 may be configured to associate between the optional treatment providers, and/or between one or more clinics providing the medicine. In some cases, the optional treatment providers can be provided by an external database, wherein said external database is configured to communicate with the medical related information module 100 in general and more specifically with the course of action database 140.

For example, the medical related information module 100 can be utilized by a computerized process configured to use third-party entities such as third-party entity 155 for the purpose of receiving data and sending data. The computerized process can be configured to use the course of action database 140 and associate among course of actions and a person, according to the healthcare agreement portfolio associated with said person. In some cases, the course of action database 140 may allow listing a plurality of courses of actions associated with costs. In such cases, the course of action database 140 may also be configured to allow a computerized process configured to use databases to list the courses of actions and sort thereof by the cost. In such an exemplary case, the course of action database 140 can also be configured to allow the computerized process to associate the sorted courses of actions to a healthcare agreement portfolio of a person.

In some cases, the course of action database 140 can be linked to external third-party entities such as third-party entity 155 for additional and/or complementary courses of actions associated with the course of action database 140. For example, the courses of actions can be groups of actions grouped in order to enable operations of healthcare services such as, schedule an appointment for an Mill test, schedule an appointment for a US test, schedule an appointment for a CT test, schedule pre-appointment for an Mill test, schedule early appointment for a ultrasound test, schedule early appointment for CT testing, schedule an appointment to a pharmacological clinic, file a request for payment from the insurance company, file a request for financial support, file a request for legal aid, and the like.

The medical related information module 100 can be defined as an abstraction embodied as a data structure and utilized by a computerized system designed for managing the reciprocal relations between the healthcare agreement portfolio associated with a person and the courses of actions to which the person is entitled, according to the healthcare agreement portfolio thereof. In some cases, the medical related information module 100 can be configured to allow changes, updates and deletion of data by a dedicated computerized system configured to change the medical related information module 100 by adding and updating the course of action database 140 and/or the healthcare-agreement database 105. In some cases, the medical related information module 100 can be implemented as a data structure, or databases held by a computerized system for the purpose of calculating, storing data, updating values, and the like. In some cases, the medical related information module 100 may implemented by two separated databases, stored and operated on two distinct computerized systems. In such cases, the medical related information module 100 can utilize a network connection such as connection 160 for communicating between the course of action database 140 and the healthcare-agreement database 105. For example, the connection 160 can be a network connection implemented over a telecommunication network. Such a telecommunication network can be, LAN, PAN, Wireless LAN, cellular phone system, and the like.

In some cases, the medical related information module 100 can be implemented as a part of a computerized system (not shown). Such a computerized system can be implemented in numerous ways, including as a process, an apparatus, a computerized system, a computer program product embodied in a computer readable storage medium utilizing a processing unit. Such a processing unit can be configured to execute instructions stored on and/or provided by a memory coupled to the processing unit. In some embodiments of the present invention the computerized system may be implemented by application containerization technology used to execute applications operating on an operating system. Such operating systems may comprise kernel-loadable modules. In some other cases, other hypervisor-based implementation may be utilized to operate the computerized system, wherein the hypervisor is functioning as a layer of software disposed between hardware and an operating system which allows the same instructions as hardware for the operating system.

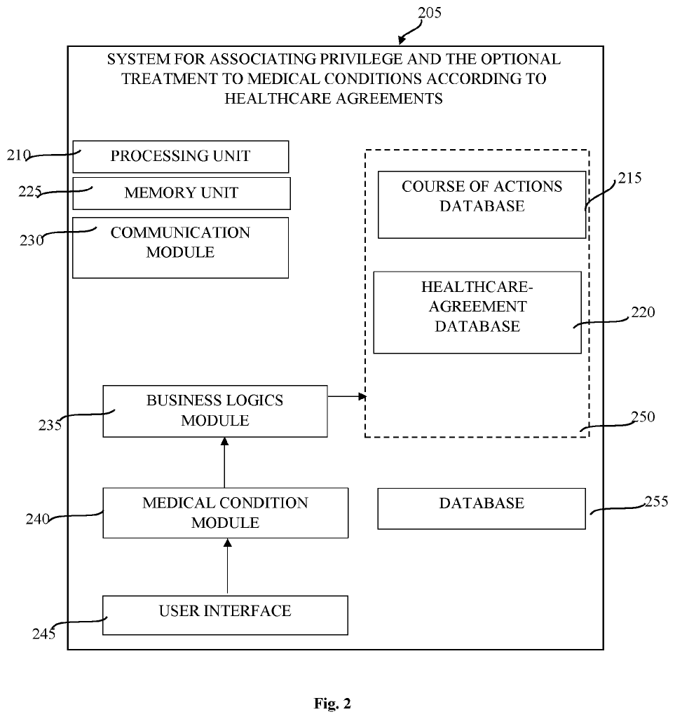

FIG. 2 discloses a computerized system for associating course of actions to medical conditions based on personal healthcare agreement portfolio, according to exemplary embodiments of the present invention. FIG. 2 shows a computerized system 205, designed to analyze healthcare agreement portfolio according to the requirements and needs derived from the medical condition of a person. Thus, the computerized system 205 can be configured to receive information comprising personal details and a medical condition representation of said person and identify the person's healthcare agreement portfolio. The computerized system 205 can also be configured to identify the course of actions corresponding with the identified healthcare agreement portfolio by matching the identified healthcare agreement portfolio with a database comprising the courses of actions associated with the categories and medical conditions. In some cases, the computerized system 205 is also configured to analyze the healthcare agreement portfolio by identify the coverage of the healthcare agreement portfolio, as elaborated below.

The computerized system 205 can comprise a user interface 245. The user interface 245 can be configured to receive medical condition representations, for the purpose of associating course of actions to medical conditions based on personal healthcare agreement portfolio. The medical condition representation provided by a person can comprise any evidence-based data indicating the medical condition of a person, as aforementioned. For example, the user interface 245 can be configured to allow a person to submit the data comprising the result of a blood test. Such submitted data can be by a typing the results to the user interface 245, uploading a text file comprising the data, providing a reference or an address of a remote computerized device configured to send data, and the like. In some cases, the user interface 245 can be arranged to direct a person to submit the data according specific a track of occurrences of data submission. In such cases, the user interface 245 can lead the person submitting the data through a specific sequence of data submissions and thereby assist the person to define the services which can address the person needs.

The computerized system 205 can also comprise medical condition module 240 designed to analyze the medical condition representation provided by the person. In some cases, the analyze process conducted by the medical condition module 240 can comprise classifying the submitted medical condition representation to classes. For example, a medical condition representation provided by the person can be classified as a blood test, an electrocardiogram test, results of electrocardiogram test, results of ultrasound test, and the like. Another example for the analyze process conducted by the medical condition module 240 can be categorizing terms, phrases, and/or word which appear in the provided medical condition representation. For example, specific terms and phrases associated with one or more medical fields can be identified in the received medical condition representation, and then be categorized accordingly.

In some embodiments of the present invention, the medical condition module 240 may be configured to work interactively with the person submitted the medical condition representation of the medical condition. For example, the medical condition module 240 can be configured to ask for additional data, for the purpose of analyzing the medical condition representation and identify the medical condition. Such additional data can be additional test results, numeric values related to the submitted tests, dates, time, and the like. In some cases, the medical condition module 240 may be configured to utilize auxiliary process or auxiliary services for preforming the analysis of the submitted medical condition representation of the medical condition. For example, the medical condition module 240 can be computerized processes which utilize algorithms, statistical models, machine learning methods and the like, to effectively analyze the medical condition representation and identify the medical condition. For example, the medical condition module 240 can utilize machine learning methods to identify the type of the medical test and categorize the medical test according to the result types. The medical condition module 240 may comprise a classification and labeling processes designed and configured to manage the classification processes and associate the category and/or the classification to the medical condition. For example, the classification and labeling process can be configured to conduct the classification process which classifies the medical condition into test types, treatment types, and the like.

In some embodiments of the present invention, the medical condition module 240 can be configured to receive data or classification by the person. In such embodiments, the medical condition module 240 can be configured to receive a questions, requests or descriptions of the medical condition. For example, a person can add a request for skin biopsy and the medical condition module 240 may receive the request for skin biopsy as the medical condition of the person. In such an exemplary case, the medical condition module 240.

The computerized system 205 can also comprise business logics module 235 designed for analyzing the healthcare agreement portfolio of the person, according to the analyzed medical condition according to the provided medical condition representation. The business logics module 235 can be configured to identify covered cases of the of the healthcare agreement portfolio of the person. Thus, the cases which are covered can be the entitlements for courses of actions, according to the healthcare agreement portfolio. The covered cases can be such as, non-covered courses of actions and thereby the courses of actions a person is not entitled for according to the agreements of the healthcare agreement portfolio, optional courses of actions, which are covered in more than one agreements of the healthcare agreement portfolio, optional courses of actions, which are covered in more than one agreements of the healthcare agreement portfolio, wherein different agreements of the healthcare agreement portfolio offering different entitlements for optional courses of actions. Such a courses of actions may be undergoing treatments, receiving medical consulting, undergoing medical tests, scheduling an appointment with a therapist, and/or taking any action required for maintenance or improvement of health via the prevention, diagnosis, and treatment.

The business logics module 235 can also be configured to communicate with a medical related information module 250, for the purpose of identifying the healthcare agreement portfolio associated with a person. In some cases, such a person may be registered in the computerized system 205. In some other cases, the business logics module 235 may comprise the details of the persons registered in the computerized system 205. The business logics module 235 can be configured to access the healthcare agreement database 220 and receive the healthcare agreements and/or the healthcare agreement portfolio of a person. In some cases, the business logics module 235 can be configured to operate in to main steps, a first step and a second step. The first step can constitute the analyzing process of the healthcare agreement portfolio of the person, according to the medical condition representation, as aforementioned. The result of the first step can be covered cases according to the healthcare agreement portfolio of a person. In some cases, the covered cases may be related to a medical condition provided by the person. For example, in case the medical condition provided by the person is classified as a bone marrow biopsy, the covered cases identified by the business logics module 235 may be related to bone marrow biopsy.

The second step of the business logics module 235 can be identifying the courses of actions according to the covered cases of the healthcare agreement portfolio and medical conditions. The second step of the business logics module 235 can be a set of computerized instructions defining a process to identify the optional courses of actions to which the person is entitled, according to the healthcare agreement portfolio thereof. In such cases, the business logics module 235 may utilize the course of actions database 215 for identifying the courses of actions corresponding to the healthcare agreement portfolio of the person whom provided the medical condition representation.

In some cases, the business logics module 235 can be configured to perform the first step and the second step and then, to output the result of the said two steps. For example, the business logics module 235 can be configured to output the result. Such an output can be to a database, a computer-readable medium, user interface application and the like. In some cases, the business logics module 235 can be configured to output a result comprising the person name, the medical condition thereof and the covered cases according to the healthcare agreement portfolio. In some cases, the business logics module 235 can be configured to output a result comprising the person name, the medical condition thereof and the optional courses of actions to which the person is entitled. In some cases, the business logics module 235 can be configured to output a result comprising the person name, the medical condition thereof and the course of actions to which the person is entitled in accordance with the medical condition thereof, and the covered cases according to the healthcare agreement portfolio.

The computerized system 205 can comprise a database 255. In some cases, such the database 255 can be configured to be utilized to store information and data, by the computerized system 205. The information and data can be the person names of the person registered in the computerized system 205, historical medical information relevant to the person registered in the computerized system 205, historical data related to the utilization of the computerized system 205, by the persons registered in the computerized system 205, and the like.

The computerized system 205 can be embodied as an application software operable on a computerized device comprising at least one memory unit such as memory unit 225, a processing unit such as processing unit 210, and a communication module 230 designed to control communication over telecommunication networks. The computerized system 205 can be configured to utilize the memory unit 225 which may be a computer-readable storage medium configured for storing data accessible by the computerized system. In some cases, the processing unit 210 may comprise at least one digital processor (not shown) instructed to execute methods and process of the computerized system 205.

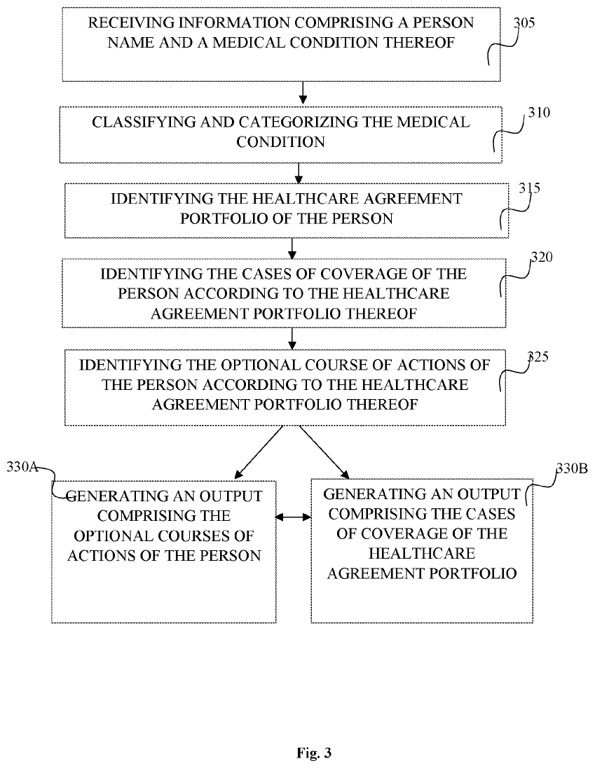

FIG. 3 shows a method configured to receive medical condition representation comprising a person name and a medical condition thereof and generating an output comprising the optional course of actions and the covered cases of the healthcare agreement portfolio, according to exemplary embodiment of the present invention. At step 305 a computerized system configured to receive information comprising a person name and a medical condition thereof receives person name and a medical condition thereof. In some cases, the medical condition representation comprises indicators, numeric values, textual descriptions, images, or any other evidence-based data indicating the medical condition of a person. In some cases, the medical condition representation can comprise a written document denoting the medical condition, or one aspect of the medical condition of a person. In some other cases, the medical condition representation can comprise a photographic evidence denoting the medical condition, or one aspect of the medical condition of a person. In some embodiments disclosed herein, the medical condition representation can comprise an auditory representation embodied as a computer-readable fashion. In some cases, a process of registration and/or authentication may be required prior providing the information. In such cases, the person providing the information may perform a process of name validation and credentials check, before providing the information. In some embodiments disclosed herein, the medical condition representation can comprise general information inputted by a user of the system, such as stress in the left knee in the past 3 days and a request for a medical appointment with a medical doctor having expertise in knee orthopedic. Diagnosis from the medical doctor having expertise in knee orthopedic can later be added into the system as part of the user's medical condition, to assist the user's medical condition, as elaborated below.

At step 310 the medical condition representation may undergo a classification and categorization phase. The categorization phase can categorize terms, phrases, and/or word which appear in the provided medical condition representation. For example, specific terms and phrases associated with one or more medical fields can be identified in the received medical condition representation, and then be categorized accordingly. In some cases, a process comprising classification of the medical condition to classes may also be carried out. For example, a medical condition representation provided by the person can be classified as a urine test, an electrocardiogram test, results of electrocardiogram test, results of ultrasound test, and the like. In some cases, the classification and categorization phase may comprise a categorization step to identify the category of the terms, phrases, and/or word which appear in the provided medical condition representation. In such cases, the categorization step can be followed by a category step to identify set of terms having some property or attribute in common and differentiated from others by kind, type, or quality. For example, the computerized system can categorize terms such as blood, blood pressure, hemoglobin, blood platelets, under the category blood. The classification step in such an exemplary case, can classify the terms categorized as blood, under the classification of “blood test”.

At step 315 the computerized system may conduct a process for identifying the healthcare agreement portfolio associated with the person associated with the medical condition. In some cases, the computerized system may utilize a grouping process designed to group some healthcare agreements of a person to a healthcare agreement portfolio. In some other cases, the computerized system can utilize metadata tags comprising keywords or terms assigned to a piece of information within the healthcare agreement stored in the agreement records, to identify a healthcare agreement portfolio of a person.

At step 320 the computerized system may conduct a process of identifying the covered cases of the person according to the healthcare agreement portfolio thereof and the provided medical condition. The computerized instructions can identify the course of actions to which the person is entitled, according to the healthcare agreement portfolio thereof. The covered cases may be such as, non-covered cases in the agreements of the healthcare agreement portfolio, discover cases which are covered in more than one agreements of the healthcare agreement portfolio, discover cases that are covered in more than one agreements of the healthcare agreement portfolio, wherein different agreements of the healthcare agreement portfolio offering different entitlements for course of actions. Such a course of actions may be undergoing treatments, receiving medical consulting, undergoing medical tests, scheduling an appointment with a therapist, and/or taking any action required for maintenance or improvement of health via the prevention, diagnosis, and treatment. In some cases, an historical information regards the historical medical activities may be utilized in the process of the covered cases. For example, in case a person registered in the computerized system is entoleted to receive a particular service a restricted number of times during a year, the computerized system may verify the number of service usages of the person, prior identifying the covered cases.

At step 325 the computerized system may identify the course of action associated with the medical condition and the healthcare agreement portfolio. The computerized system can be configured to associate among optional course of actions and a person, according to the healthcare agreement portfolio associated with said person and the medical condition. In some cases, the optional course of actions may allow listing a plurality of optional course of actions associated with costs. In such cases, the course of action may be listed and sorted by the computerized system according to cost. In such an exemplary case, the computerized system may associate the sorted optional course of actions to a healthcare agreement portfolio of a person. In some cases, other parameters utilized to sort the optional course of actions may be utilized by the computerized system. The parameters utilized to sort the optional course of actions can be such, availability of services the person may be required to use, urgency level pointed by the person, location, service quality, and the like. In some cases, an historical information regards the historical medical activities may be utilized in the process of the course of actions. For example, in case a person registered in the computerized system is entoleted to receive a particular service a restricted number of times during a year, the computerized system may verify the number of service usages of the person, prior identifying the course of actions.

At step 330A the computerized system may output some of the results. Such an output can be to a database, a computer-readable medium, user interface application and the like. In some cases, the output of the result can comprise the person name, the output of the result can comprise the person name, the medical condition thereof and the course of actions to which the person is entitled. At step 330B the computerized system may output some of the results. Such an output can be to a database, a computer-readable medium, user interface application and the like. In some cases, the output of the result can comprise the person name, the medical condition thereof and the covered cases according to the healthcare agreement portfolio.

In some embodiments of the present invention, the computerized system may preform step 33 OB first and then halt without preforming step 330A. In possible embodiments of the present invention, the computerized system may preform step 330A first and then halt without preforming step 330B. In possible embodiments of the present invention, the computerized system may preform step 33 OB and then preform step 330A. In possible embodiments of the present invention, the computerized system may preform step 330A and then preform step 330B.

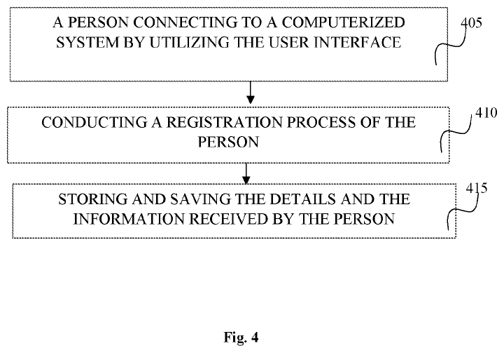

FIG. 4 shows a registration process of a person, to the computerized system, according to exemplary embodiments of the present invention. At step 405 a person utilizes a user interface of a computerized system configured to receive information comprising a person name and a medical condition thereof. The person utilizing the user interface may operate a computerized device such as personal computer, a tablet person computer, a mobile telephone and the like. At step 410 the computerized system may conduct a registration process. In such a process the person registering to the computerized system may provide the name thereof. In some cases, additional information may be provided by the person. The information can comprise, user name, password, other credentials, power of attorney agreement, and the like. At step 415 the detailed provided by the person are stored by the computerized system. In some cases, the detailed provided by the person may be sent to a third-party entity for the purpose of receiving data and sending data. In some cases, such as third-party entity can be used for receiving additional and/or complementary course of actions associated with the course of actions identified by the computerized system.

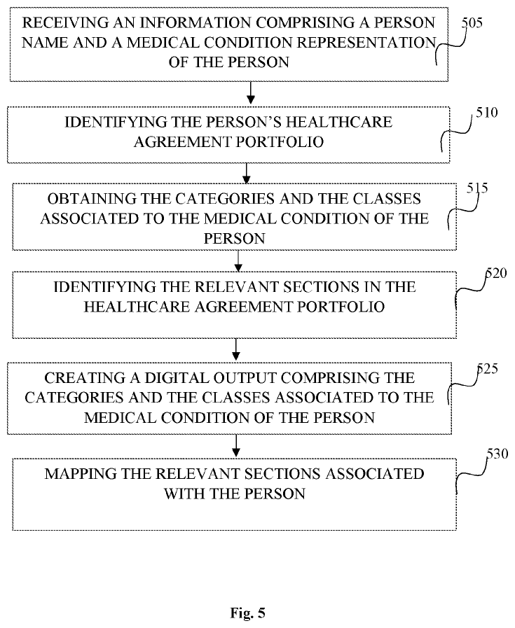

FIG. 5 shows a process of identifying covered cases in a healthcare agreement portfolio, according to exemplary embodiments of the present invention. At step 505 the computerized system may receive an information comprising a person name and a medical condition representation thereof. In some cases, the medical condition representation may be classified and categorized as aforementioned. At step 510 the computerized system may identify the person's healthcare agreement portfolio. In some cases, wherein a healthcare agreement portfolio cannot be identified, the computerized system lists the healthcare agreements associated with the person and generate an ad-hoc healthcare agreement portfolio. In some cases, such an ad-hoc healthcare agreement portfolio may be a process of grouping the healthcare agreements associated with the person and exist in a database controlled by the computerized system. At step 515 the computerized system obtains the categories and the classes associated to the medical condition of the person.

At step 520 the computerized system identifies the relevant sections in the healthcare agreement portfolio which are related to the categories and the classes associated to the medical condition of the person. The relevant sections can be sections, paragraphs, portions of text, images and other information types, associated with the healthcare agreement portfolio. Thus, the computerized system may identify the relevant sections in the healthcare agreements which can cover the categories and the classes associated to the medical condition of the person. For example, the computerized system may identify one or more clauses which comprises information relevant to the categories and the classes associated to the medical condition of the person. In some cases, the computerized system may identify more than one relevant section in more than one healthcare agreements in the person's healthcare agreement portfolio, which care relevant to the categories and the classes associated to the medical condition of the person.

In some cases, the computerized system may utilize some metadata for identifying the relevant sections. The metadata, i.e., metadata tags, can comprise keywords or terms assigned to a piece of information within the healthcare agreements of the healthcare agreement portfolio. At step 525 the computerized system maps the relevant sections identified in step 520 and generates a digital output comprising the information the person's name, the categories and the classes associated to the medical condition of the person, and the healthcare agreement portfolio. The system may also identify the covered cases within the healthcare agreement portfolio and include the identified covered cases in the digital output. In some cases, such a digital output can be a computer-readable data, structured and configured to be read by a computerized system. For example, the computer-readable data can be a digital file, an entry in a digital database, and assembly of data units which can be read, stored, manipulated, utilized by a computerized device or a computerized system, and the like. At step 530 the computerized system can output the map of the relevant sections associated with the person for the purpose of presentation. In some cases, such a map can be utilized to identify covered cases within the healthcare agreement portfolio.

While the disclosure has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the disclosed subject matter not be limited to the particular embodiment disclosed as the best mode contemplated for carrying out this invention, but only by the claims that follow.

What is claimed is:

1. A method operable on a computerized system for associating privilege and an optional treatment to medical conditions according to healthcare agreements, comprising:
receiving a medical condition of a person represented as a record of data, wherein the record of data comprises the medical condition thereof, wherein the medical condition states one or more aspects of the health of the person;
categorizing the record of data representing the medical condition, wherein the categorizing process categorizes terms, and/or phrases, and/or word which appear in the record of data representing the medical condition;
identifying a healthcare agreement portfolio associated with the person associated with the record of data representing the medical condition;
identifying relevant sections in the healthcare agreement portfolio which are related to categories identified in the record of data representing the medical condition;
identifying course of actions to which the person is entitled, according to the identified relevant sections in the healthcare agreement portfolio which are related to the categories identified in the record of data representing the medical condition thereof;
generating a digital output comprising the person's name, the categories associated to the medical condition of the person, and the course of actions to which the person is entitled, according to the identified relevant sections in the healthcare agreement portfolio.

2. The method of claim 1, wherein generating a digital output further comprises associating the covered cases, corresponding with the identified healthcare agreement portfolio.

3. The method of claim 2, wherein the covered cases are sets of at least one course of action.

4. The method of claim 1, wherein the record of data further comprises the name of the person.

5. The method of claim 1, further comprises a classifying process to identify classes in the record of data representing the medical condition to classes.

6. The method of claim 5, wherein the classes are identified sets of terms having some property or attribute in common and differentiated from others by kind, type, or quality.
